(19) United States
(12) Patent Application Publication
(10) Pub. No.: US 2023/0035202 A1
(43) Pub. Date: Feb. 2, 2023

(54) PROCESS FOR PREPARING PARTICLES COATED WITH SILICON OXIDE BY FLAME SPRAY PYROLYSIS

(71) Applicant: L'OREAL, Paris (FR)

(72) Inventors: Valérie JEANNE-ROSE, AULNAY-SOUS-BOIS (FR); Henri SAMAIN, CHEVILLY-LARUE (FR); Yiannis DELIGIANNAKIS, IOANNINA (GR); Maria LOULOUDI, IOANNINA (GR)

(73) Assignee: L'OREAL, Paris (FR)

(21) Appl. No.: 17/789,496

(22) PCT Filed: Dec. 24, 2020
(86) PCT No.: PCT/EP2020/087874

§ 371 Date: Jun. 27, 2022

(30) Foreign Application Priority Data
Dec. 27, 2019 (FR) 1915679

(51) Int. Cl. C01B 13/34; A61K 8/02; A61K 8/25; A61K 8/27; A61K 8/29; A61Q 17/04; C01G 23/04; C01G 9/02

(52) U.S. Cl. CPC C01B 13/34; A61K 8/25; A61K 8/27; A61K 8/29; A61K 8/0241; A61Q 17/04; C01G 9/02; C01G 23/04

(57) ABSTRACT

The present invention relates to a process for preparing oxide particles, in particular metal oxide particles, coated with silicon oxide by means of flame spray pyrolysis technology, to oxide particles, in particular metal oxide particles, coated with silicon oxide, and to a composition comprising said particles. The present invention also relates to specific oxide particles, in particular metal oxide particles, coated with silicon oxide derived from such a process, to the compositions comprising such particles and also to the uses thereof.

The present invention relates to a process for preparing oxide particles, in particular metal oxide particles, coated with silicon oxide by means of flame spray pyrolysis technology, to oxide particles, in particular metal oxide particles, coated with silicon oxide, and to a composition comprising said particles.

The present invention also relates to specific oxide particles, in particular metal oxide particles, coated with silicon oxide derived from such a process, to the compositions comprising such particles and also to the uses thereof.

Mineral compounds, also called oxides, such as zinc, copper or iron oxide, are used in many applications (cosmetics, paints, stains, electronics, rubber, etc.). By way of example, zinc oxide is notably used for its optical properties, in particular its light absorption and/or light scattering properties which make it possible to protect surfaces from UV radiation and/or to convert ambient light into electricity.

However, some of these oxides have the drawback of being particularly unstable over time. They notably have a tendency to degrade in the presence of water originating from the composition comprising them or from atmospheric moisture. Such a degradation leads to a partial or even total solubilization of the oxide in water and has the effect of greatly reducing, or even removing, the desired properties of said oxide.

These degradations may prove particularly problematic in certain situations, and notably when the product is for public use such as in cosmetic products, paints or the food industry. As examples, the ultraviolet radiation protection of the sun compositions reduces as the zinc oxide degrades. In order to overcome these drawbacks, it is customary to package these compounds in anhydrous mediums, or even to protect them with specific packaging, which proves particularly restrictive.

Other approaches, such as the preparation of an extemporaneous mixture at the moment of use or the addition of preservatives that make it possible to stabilize these compounds do not represent satisfactory solutions either, both from the point of view of consumers, and of the environment. But reducing the preservative compounds leads to more acidic formulations being used, thus forming conditions that are more unstable for the oxides used. Magnesia or zinc, copper and iron oxides are notably sensitive to this acidity.

In certain applications, the oxide compound is intended to be used in particulate form, such as for example the application of a fluid formulation onto a surface. There is then a risk of the particles evolving with the ambient air, formed of CO₂ and water. If furthermore, the surface on which the fluid formulation is applied comprises water or if it may be subsequently be brought into contact with water, the degradation of the oxide compound will be accelerated. By way of example, perspiration produces water, in general acidic water, and the latter may degrade the oxide compound present in cosmetic compositions. Likewise when the application of a cosmetic product onto the skin or hair is followed by a desired or undesired (rain, spray, etc.) supply of water. Thus, the use of an anhydrous product containing such oxides may lead to a degradation of the oxide once the product has been applied to the skin or hair.

These drawbacks are even greater when fine particles are used. However, such particles are commonly used in cosmetics due to the fact that they are invisible and undetectable by touch.

In order to protect the metal oxide particles, it is known to coat them with a fatty or polymeric coating. However, the use of a fatty coating limits the use of the particles and requires the use of hydrophobic products or surfactants. Furthermore, the production of a polymeric coating also poses problems owing to the limits put on microplastics.

It had been envisaged to coat the metal oxides with silica by means of sol-gel processes. However, this solution is not entirely satisfactory. The protection of the metal oxide has proved particularly poor in an acid medium.

It is also known to use a flame spray pyrolysis method (FSP method) to prepare zinc oxide particles.

Flame spray pyrolysis or FSP is a well-known method these days, which was essentially developed for the synthesis of ultrafine powders of single or mixed oxides of various metals (e.g. SiO₂, Al₂O₃, B₂O₃, ZrO₂, GeO₂, WO₃, Nb₂O₅, SnO₂, MgO, ZnO), with controlled morphologies, and/or the deposition thereof on various substrates, by starting from a wide variety of metal precursors, generally in the form of organic or inorganic, preferably inflammable, sprayable liquids; the liquids sprayed into the flame, by being burnt, notably emit nanoparticles of metal oxides which are sprayed by the flame itself onto these various substrates. This method has also been used to manufacture oxide particles covered with a layer of silica. The principle of this method has been recalled for example in the recent (2011) publication by Johnson Matthey entitled "Flame Spray Pyrolysis: a Unique Facility for the Production of Nanopowders", Platinum Metals Rev., 2011, 55, (2), 149-151. Numerous variants of FSP processes and reactors have also been described, by way of example, in the patents or patent applications: US 5 958 361, US 2 268 337, WO 01/36332 or US 6 887 566, WO 2004/005184 or US 7 211 236, WO 2004/056927, WO 2005/103900, WO 2007/028267 or US 8 182 573, WO 2008/049954 or US 8 231 369, WO 2008/019905, US 2009/0123357, US 2009/0126604, US 2010/0055340, WO 2011/020204.

However, the layer of silica thus formed has nevertheless proven too thin and insufficient to protect the oxide from water.

There is therefore a real need to develop a process for preparing oxide particles, and notably metal oxide particles, making it possible to give said particles a good stability over time, and very particularly a good water resistance, while preserving the properties of the oxide used, such as good optical properties in terms of absorption and/or scattering of light, more particularly of ultraviolet radiations.

These aims are achieved by the present invention, one subject of which is notably a process for preparing coated particles of element M oxide comprising at least the following steps:
a) preparing a composition (A) by adding one or more element M precursors to one or more combustible solvents; then
b) in a flame spray pyrolysis device, forming a flame by injecting the composition (A) and an oxygen-containing gas until aggregates of element M oxide are obtained; and
c) injecting into the flame a composition (B) comprising one or more silicon precursors and one or more polar protic solvents other than water until an inorganic coating layer containing a silicon oxide is obtained on the surface of said aggregates of element M;
it being understood that:
said element M is chosen from alkali metals from column 1, alkaline-earth metals from column 2 and the elements from columns 3 to 16 of the Periodic Table of the Elements, and elements from the family of lanthanides, and
the silicon precursor(s) comprise at least two silicon atoms and several Si-carbon covalent bonds.

It has been observed that the process according to the invention makes it possible to obtain particles of a specific element M oxide which are coated with a layer of silicon oxide, are particularly stable over time and have a good resistance to water, even at acid pH.

More particularly, the process of the invention makes it possible to form a layer of silicon dioxide having a specific "4-membered ring" structure. This specific assembly of silicon dioxide encloses the element M oxide thus forming a protective layer around this compound.

Furthermore, unlike conventional coating processes, the process according to the invention has the advantage, despite the presence of the coating, of retaining good intrinsic properties of the centre. Indeed, owing to the specific nature of the coating layer, it is possible for a given particle weight, to reduce the proportion of metal oxide, without however reducing and/or negatively affecting the properties of said metal oxide.

Thus, the process of the invention makes it possible to produce stable metal oxide particles, while avoiding the inconveniences owing to the increase in the amount of particles which would be conventionally necessary in order to maintain the good optical properties of said oxides.

Furthermore, the compositions comprising coated metal oxide particles may protect fillers, pigments, or other water-sensitive inorganic active agents for example magnesium oxide.

By varying the quality of the metal oxides, notably the silica, it is possible to obtain a water protection that is high, but that is not complete, an intermediate protection. This enables, for example, a more gradual or controlled release of the metal oxide from the centre.

These particles of element M oxide comprise a core (1) and one or more upper coating layers (2) covering said core (1), and are characterized in that:
(i) the core (1) consists of one or more element M oxides, preferably in the crystalline state;
(ii) said upper coating layer(s) (2) cover at least 90% of the surface of the core (1), preferably cover the whole of the surface of the core (1), and comprise one or more silicon oxides;
(iii) said element M is chosen from magnesium, calcium, zinc, copper, iron, zirconium, aluminium, gallium, indium, tin, scandium, yttrium, lanthanum, cerium, praseodymium, promethium, samarium, europium, gadolinium, terbium, dysprosium, holmium, erbium, thulium, ytterbium and lutecium, and
(iv) the (M/Silicon)_{particle} molar atomic ratio is within the range of from 0.1 to 10, preferably from 0.2 to 2, and more preferentially from 0.5 to 1.5.

More particularly, the particles of specific element M oxide according to the invention only deteriorate very little over time in the presence of water, even when they are formulated in an aqueous composition, or even an acid composition.

It has also been observed that the particles prepared according to the invention retain the properties intrinsic to the element M oxide used, such as good optical properties in terms of light absorption and/or light scattering. More particularly, they have a high UV absorption and a low visible scattering or a high visible scattering, then allowing uses such as sun protection and/or modification of the visual appearance, while benefiting from resistance in the presence of water.

Moreover, the compositions comprising such particles have shown a good screening power, notably with respect to long and short UV-A radiation.

Furthermore, the compositions comprising the particles of the invention have an especially high transparency, which may prove advantageous when the composition is applied then left to dry on the coating, and in particular on the skin.

Moreover, since the particles of element M oxide that are coated with silicon oxide according to the invention do not require a hydrophobic coating, it is possible to use them over a broad formulations spectrum (for example, in entirely aqueous formulations and/or surfactant-free formulations). When the formulations thus obtained end up in water (washbasin drainage, lake or sea), the risk of inappropriate deposit (on the edges of the washbasin, on the walls of the pipes or on rocks) is furthermore reduced.

### BRIEF DESCRIPTION OF THE FIGURES

The attached drawings are schematic. The drawings are not necessarily to scale; they are above all intended to illustrate the principles of the invention.

FIG. 1 represents a cross-sectional view of a zinc oxide particle according to one embodiment of the invention.

Other subjects, features, aspects and advantages of the invention will emerge even more clearly on reading the description and the example that follows. In the present description, and unless otherwise indicated:
the expression "at least one" is equivalent to the expression "one or more" and can be replaced therewith;
the expression "between" is equivalent to the expression "ranging from" and can be replaced therewith, and implies that the limits are included;
the expression "keratin materials" denotes in particular the skin and also human keratin fibres such as the hair;
the core (1) is also referred to as the "centre";
the upper coating layers (2) are also referred to as "outer layers", "shell" or "coating";
the expression "(in)organic compounds" is equivalent to "organic or inorganic compounds";
an "alkyl" is understood to mean an "alkyl radical", i.e. a C₁ to C₁₀, particularly C₁ to C₈, more particularly C₁ to C₆, and preferentially C₁ to C₄, linear or branched hydrocarbon-based radical, such as methyl, ethyl, propyl, isopropyl, n-butyl, isobutyl or tert-butyl;
an "aryl" radical is understood to mean a monocyclic or fused or non-fused polycyclic carbon-based group, comprising from 6 to 22 carbon atoms, at least one ring of which is aromatic; preferentially, the aryl radical is a phenyl, biphenyl, naphthyl, indenyl, anthracenyl or tetrahydronaphthyl, preferably a phenyl;
an "arylate" radical is understood to mean an aryl group which comprises one or more —C(O)O^{—} carboxylate groups, such as naphthalate or naphthenate;
"complexed zinc" is understood to mean that the zinc forms a "metal complex" or "coordination compounds" in which the metal ion, corresponding to the central atom, i.e. the zinc, is chemically bonded to one or more electron donors (ligands);
a "ligand" is understood to mean a coordinating organic chemical group or compound, i.e. which comprises at least one carbon atom and which is capable of coordinating with a metal, notably the Zn atom, preferably Zn(II) and which, once coordinated or complexed, results in metal compounds corresponding to principles of a coordination sphere with a predetermined number of electrons (internal complexes or chelates) - see Ullmann's Encyclopedia of Industrial Chemistry, "Metal complex dyes", 2005, p. 1-42. More particularly, the ligand(s) are organic groups which comprise at least one group that is electron-donating via an inductive and/or mesomeric effect, more particularly bearing at least one amino, phosphino, hydroxy or thiol electron-donating group, or the ligand is a persistent carbene, particularly of "Arduengo" type (imidazol-2-ylidenes) or comprises at least one carbonyl group. As ligand, mention may more particularly be made of: i) those which contain at least one phosphorus atom -P< i.e. phosphine such as triphenyl phosphines; ii) bidendate ligands of formula R—C(X)—CR'R"—C(X)—R''' with R and R'''', which are identical or different, representing a linear or branched (C₁-C₆)alkyl group, and R' and R", which are identical or different, representing a hydrogen atom or a linear or branched (C₁-C₆)alkyl group, preferentially R' and R" represent a hydrogen atom, X represents an oxygen or sulfur atom, or an N(R) group with R representing a hydrogen atom or a linear or branched (C₁-C₆)alkyl group, such as acetylacetone or β-diketones; iii) (poly)hydroxy carboxylic acid ligands or formula [HO—C(O)]ₙ—A—C(O)—OH and the deprotonated forms thereof with A representing a monovalent group when n has the value zero or a polyvalent group when n is greater than or equal to 1, which is saturated or unsaturated, cyclic or non-cyclic and aromatic or non-aromatic based on a hydrocarbon comprising from 1 to 20 carbon atoms which is optionally interrupted by one or more heteroatoms and/or is optionally substituted, notably with one or more hydroxyl groups: preferably, A represents a monovalent (C₁-C₆)alkyl group or a polyvalent (C₁-C₆)alkylene group optionally substituted with one or more hydroxyl groups; and n representing an integer between 0 and 10 inclusive; preferably, n is between 0 and 5, for instance among 0, 1 or 2; such as lactic, glycolic, tartaric, citric and maleic acids, and arylates such as naphthalates; and iv) C₂ to C₁₀ polyol ligands, comprising from 2 to 5 hydroxyl groups, notably ethylene glycol, glycerol, more particularly still the ligand(s) bear a carboxy, carboxylate or amino group, particularly the ligand is chosen from acetate, (Ci-C₆)alkoxylate, (di)(C₁-C₆)alkylamino, and arylate, such as naphthalate or naphthenate, groups;

The term "fuel" is understood to mean a liquid compound which, with dioxygen and energy, is burnt in a chemical reaction generating heat: combustion. In particular the liquid fuels are chosen from protic solvents, in particular alcohols such as methanol, ethanol, ispropanol, n-butanol; aprotic solvents in particular chosen from esters such as methyl esters and those derived from acetate, such as 2-ethylhexyl acetate, acids such as 2-ethylhexanoic acid (EHA), acyclic ethers such as ethyl ether, methyl tert-butyl ether (MTBE), methyl tert-amyl ether (TAME), methyl tert-hexyl ether (THEME), ethyl tert-butyl ether (ETBE), ether tert-amyl ether (TAEE), diisopropyl ether (DIPE), cyclic ethers such as tetrahydrofuran (THF), aromatic hydrocarbon or arenes such as xylene, non-aromatic hydrocarbons; and mixtures thereof. The fuels may optionally be chosen from liquified hydrocarbons such as acetylene, methane, propane or butane; and mixtures thereof.

The term "pigment" is understood to mean any inorganic pigment, of synthetic or natural origin, which gives colour to keratin materials. The solubility of the pigments in water at 25° C. and at atmospheric pressure (760 mmHg) is less than 0.05 % by weight, and preferably less than 0.01%.

They are white or coloured solid particles which are naturally insoluble in the hydrophilic and lipophilic liquid phases usually employed in cosmetics. More particularly, they are pigments with little or no solubility in aqueous-alcoholic media.

The pigments that may be used are notably chosen from the mineral pigments known in the art, notably those described in Kirk-Othmer's Encyclopedia of Chemical Technology and in Ullmann's Encyclopedia of Industrial Chemistry. Pigments that may notably be mentioned include inorganic pigments such as those defined and described in Ullmann's Encyclopedia of Industrial Chemistry "Pigment organics", 2005 Wiley-VCH Verlag GmbH & Co. KGaA, Weinheim and ibid, "Pigments, Inorganic, 1. General" 2009 Wiley-VCH Verlag GmbH & Co. KGaA, Weinheim.

These pigments may be in powder form. The pigments may be chosen, for example, from mineral pigments, pigments with special effects such as nacres or glitter flakes, and mixtures thereof.

The pigment may be a mineral pigment. The term "mineral pigment" is intended to mean any pigment that satisfies the definition in Ullmann's Encyclopedia in the chapter on inorganic pigments. Among the mineral pigments that are useful in the present invention, mention may be made of iron oxides, chromium oxides, manganese violet, ultramarine blue, chromium hydrate, ferric blue and titanium oxide. The pigment(s) may also be pigments with special effects.

The term "pigments with special effects" refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semitransparent or transparent shade.

Examples of pigments with special effects that may be mentioned include nacreous pigments such as titanium mica coated with an iron oxide, mica coated with an iron oxide, mica coated with bismuth oxychloride, titanium mica coated with chromium oxide, titanium mica coated with a dye notably of the abovementioned type, and also nacreous pigments based on bismuth oxyhalide, such as bismuth oxychloride.

The nacres may more particularly have a yellow, pink, red, bronze, orange, brown, gold and/or coppery colour or tint.

As illustrations of nacres that may be used in the context of the present invention, mention may notably be made of the gold-coloured nacres sold notably by the company Engelhard under the name Gold 222C (Cloisonne), Sparkle gold (Timica), Gold 4504 (Chromalite) and Monarch gold 233X (Cloisonne); the bronze nacres sold notably by the company Merck under the names Bronze fine (17384) (Colorona) and Bronze (17353) (Colorona), by the company Eckart under the name Prestige Bronze and by the company Engelhard under the name Super bronze (Cloisonne); the orange nacres sold notably by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold notably by the company Engelhard under the names Nu-antique copper 340XB (Cloisonne) and Brown CL4509 (Chromalite); the nacres with a copper tint sold notably by the company Engelhard under the name Copper 340A (Timica) and by the company Eckart under the name Prestige Copper; the nacres with a red tint sold notably by the company Merck under the name Sienna fine (17386) (Colorona); the nacres with a yellow tint sold notably by the company Engelhard under the name Yellow (4502) (Chromalite); the red-tinted nacres with a golden tint sold notably by the company Engelhard under the name Sunstone G012 (Gemtone); the black nacres with a golden tint sold notably by the company Engelhard under the name Nu-antique bronze 240 AB (Timica); the blue nacres sold notably by the company Merck under the names Matte blue (17433) (Microna), Dark Blue (117324) (Colorona); the white nacres with a silvery tint sold notably by the company Merck under the name Xirona Silver; and the golden-green pinkish-orange nacres sold notably by the company Merck under the name Indian summer (Xirona), and mixtures thereof.

In addition to nacres on a mica support, multilayer pigments based on synthetic substrates such as alumina, silica, sodium calcium borosilicate or calcium aluminium borosilicate, and aluminium, may be envisaged.

### The Process for Preparing the Particles Coated With Silicon Oxide

The preparation process according to the invention comprises a step (a) of preparing a composition (A) containing one or more element M precursors and one or more combustible solvents; said element M being chosen from alkali metals from column 1, alkaline-earth metals from column 2, the elements from columns 3 to 16 of the Periodic Table of the Elements, and elements from the family of lanthanides.

Advantageously, the element M is chosen from magnesium, calcium, zinc, copper, iron, titanium, zirconium, aluminium, gallium, indium, tin, scandium, yttrium, lanthanum, cerium, praseodymium, promethium, samarium, europium, gadolinium, terbium, dysprosium, holmium, erbium, thulium, ytterbium and lutecium; preferably from magnesium, calcium, zinc, copper, iron, titanium, aluminium, tin, lanthanum, cerium and yttrium.

The element M precursor(s) preferably comprise one or more atoms of element M optionally complexed with one or more ligands containing at least one carbon atom, and more preferentially optionally complexed with one or more ligands containing at least two carbon atoms.

Preferably, the ligand(s) are chosen from acetate, (C₁-C₆)alkoxylate, (di)(Ci-C₆)alkylamino, and arylate, such as naphthalate or naphthenate, groups.

The combustible solvents that can be used according to the invention may be chosen from the combustible solvents conventionally used in flame spray pyrolysis.

Preferably, the combustible solvent(s) are chosen from protic combustible solvents, aprotic combustible solvents, and mixtures thereof; more preferentially from alcohols, esters, acids, acyclic ethers, cyclic ethers, aromatic hydrocarbon or arenes, non-aromatic hydrocarbons, and mixtures thereof; and better still from 2-ethylhexyl acetate, 2-ethylhexanoic acid (EHA), ethyl ether, methyl tert-butyl ether (MTBE), methyl tert-amyl ether (TAME), methyl tert-hexyl ether (THEME), ethyl tert-butyl ether (ETBE), ether tert-amyl ether (TAEE), diisopropyl ether (DIPE), tetrahydrofuran (THF), xylene, and mixtures thereof.

More preferentially, the combustible solvent(s) are chosen from aprotic combustible solvents comprising at least three carbon atoms; more preferentially still from xylene, tetrahydrofuran, 2-ethylhexyl acetate, 2-ethylhexanoic acid (EHA), and mixtures thereof.

Advantageously, the content of element M precursor in composition (A) is between 1% and 60% by weight and preferably between 15% and 30% by weight relative to the total weight of composition (A).

The preparation process according to the invention further comprises a step (b) of injecting composition (A), prepared in step (a), and an oxygen-containing gas into a flame spray pyrolysis (FSP) device to form a flame.

During this step (b), composition (A) and the oxygen-containing gas are advantageously injected into the flame spray pyrolysis device, by two injections that are separate from one another. In other words, composition (A) and the oxygen-containing gas are injected separately, i.e. composition (A) and the oxygen-containing gas are not injected by means of a single nozzle.

More particularly, composition (A) is transported by one tube, whereas the oxygen-containing gas (also referred to as "dispersion Oxygen") is transported by another tube. The inlets of the two tubes are arranged so that the oxygen-containing gas produces a negative pressure and, via a Venturi effect, causes the composition (A) to be sucked up and converted into droplets.

Step (b) may optionally further comprise an additional injection of a “premix” mixture comprising oxygen and one or more combustible gases. This "premix" mixture is also referred to as a "supporting flame oxygen" and enables the production of a support flame intended to ignite and maintain the flame resulting from composition (A) and the oxygen-containing gas (i.e. "dispersion Oxygen").

Preferably, during step (b), composition (A), the oxygen-containing gas and optionally the "premix" mixture when it is present, are injected into a reaction tube, also referred to as an "enclosing tube". Preferably, this reaction tube is made of metal or of quartz. Advantageously, the reaction tube has a height of greater than or equal to 30 cm, more preferentially greater than or equal to 40 cm, and better still greater than or equal to 50 cm. Advantageously, the length of said reaction tube is between 30 cm and 300 cm, preferably between 40 cm and 200 cm, more preferentially between 45 cm and 100 cm, and better still this length is equal to 50 cm.

The weight ratio of the mass of solvent(s) present in composition (A) on the one hand, to the mass of oxygen-containing gas on the other hand, is defined as follows: Firstly, the amount of oxygen-containing gas (also referred to as oxidizer compound) is calculated in order for the assembly formed by composition (A), i.e. the combustible solvent(s) and the zinc precursor(s) on the one hand, and the oxygen-containing gas on the other hand, to be able to react together in a combustion reaction in a stoichiometric ratio (therefore without an excess or deficit of oxidizer compound).

Starting from this calculated amount of oxygen-containing gas, also referred to as "calculated oxidizer", a new calculation is performed to deduce therefrom the amount of oxygen-containing gas to be injected, also referred to as "oxidizer to be injected", according to the formula: Oxidizer to be injected = Calculated oxidizer / φ with φ preferably between 0.30 and 0.9, and more preferentially between 0.4 and 0.65.

This method is notably defined by Turns, S. R. in An Introduction to Combustion: Concepts and Applications, 3rd ed.; McGraw-Hill: New York, 2012.

Step (b) of the preparation process according to the invention makes it possible to obtain aggregates of element M oxide. Preferably, the element M oxide thus formed is stable. The term "stable" is understood to mean the oxide higher than the metal will take in an oxidizing medium. As examples, when an iron precursor is used, the oxide obtained is Fe₂O₃, and not Fe₃O₄. Likewise, when a copper precursor is used, the oxide obtained is CuO, and not Cu₂O.

More preferentially, the element M oxide has the formula MₓO_{y}, with x and y such that 1 ≤ y/x ≤ 2.

According to one specific embodiment of the invention, the element M is chosen from alkaline-earth metals from column 2 of the Periodic Table of the Elements, and the element M oxide thus formed has the formula MO. In other words, according to this embodiment, x=y=1.

According to yet another specific embodiment of the invention, the element M is chosen from the elements from columns 3 to 16 of the Periodic Table of the Elements, and elements from the family of lanthanides; and more particularly from the elements from columns 3 and 4, elements from the family of lanthanides, the elements from column 8 and the elements from columns 11 to 14.

According to this embodiment, the oxide(s) of element M thus formed are preferably chosen from zinc oxide ZnO, magnesium oxide MgO, calcium oxide CaO, copper oxide CuO, titanium oxide TiO₂, iron oxide Fe₂O₃, aluminium oxide Al₂O₃, cerium oxide CeO₂, lanthanum oxide La₂O₃ and yttrium oxide Y₂O₃; and more preferentially from zinc oxide ZnO, magnesium oxide MgO, calcium oxide CaO, copper oxide CuO, titanium oxide TiO₂ and iron oxide Fe₂O₃.

The preparation process according to the invention further comprises a step (c) of injecting, into the flame formed during step (b), a composition (B) comprising one or more silicon precursors and one or more polar protic solvents other than water; the silicon precursor(s) comprising at least two silicon atoms and several Si-carbon covalent bonds.

In other words, the process of the invention is continuous and the flame formed in step (b) is maintained.

During step (c) of the preparation process of the invention, the compositions (A) and (B) are injected separately and simultaneously. In other words, composition (A) is transported by one tube, whereas composition (B) is transported by another tube. The distance between the outlet of the two tubes is preferably at least 30 cm, and more preferentially at least 40 cm.

Preferably, the flame formed during step (b) is at a temperature above or equal to 2000° C., in at least one part of the flame.

At the site of the injection of the composition (B) into the flame formed in step (b) and maintained in step (c), i.e. during step (c), the temperature is preferably between 200° C. and 600° C., and more preferentially between 300° C. and 400° C.

Advantageously, during step c), composition (B) is injected via a spraying ring, placed above said reaction tube as described above, where in particular the injection of composition (A) takes place.

The silicon precursor(s) in composition (B) comprise at least two silicon atoms and several Si-carbon covalent bonds, and preferably at least three silicon atoms and several Si-carbon covalent bonds.

Advantageously, the silicon precursor(s) are chosen from hexadimethyldisiloxane, 1,2-bis(triethoxysilyl)ethane, 1,2-bis(trimethoxysilyl)ethane, and mixtures thereof.

Preferably, the silicone oxide thus formed is silicon dioxide SiO₂, and the latter is more preferentially in a "4-membered ring" structure.

During the process according to the invention, a (M/Silicon)_{injected} molar atomic ratio can be calculated. This ratio corresponds to the amount in moles of element M atoms injected during step (b) on the one hand, to the amount in moles of silicon atoms injected during step (c) on the other hand. Preferably, this (M/Silicon)_{injected} molar atomic ratio is within the range of from 0.1 to 10, more preferentially from 0.2 to 2, and more preferentially from 0.5 to 1.5.

Preferably, nitrogen is bubbled into composition (B) of the invention, prior to its injection during step (c). The rate of injection of composition (B) can then be controlled by a determination of the pressure known by a person skilled in the art, for instance the method defined by Scott, D.W.; Messerly, J.F.; Todd, S.S.; Guthrie, G.B.; Hossenlopp, I.A.; Moore, R.T.; Osborn, A.G.; Berg, W.T.; McCullough, J.P., Hexamethyldisiloxane: chemical thermodynamic properties and internal rotation about the siloxane linkage, J. Phys. Chem., 1961, 65, 1320-6.

According to one specific embodiment of the invention, composition (B) as described above is, prior to its injection during step (c), brought to a temperature within the range extending from 25° C. to 70° C., more preferentially from 30° C. to 60° C.

Preferably, the content of silicon precursor in composition (B) injected during step (c) of the process according to the invention is between 1% and 60% by weight, more preferentially between 5% and 30% by weight, relative to the total weight of the composition (B).

Advantageously, the polar protic solvent(s) other than water, present in composition (B), are chosen from (C₁-C₈)alkanols. More preferentially, composition (B) comprises ethanol.

Preferably, the polar protic solvent(s) other than water, present in composition (B), are chosen from solvents that are combustible at the flame temperature of step (c), preferably combustible at a temperature between 200° C. and 600° C.; and more preferentially between 300° C. and 400° C. Better still, the polar protic solvent(s) other than water, present in composition (B), have a boiling point above or equal to room temperature (25° C.), and more preferentially between 50° C. and 120° C.

Preferably, the content of polar protic solvent(s) other than water present in composition (B) is between 40% and 99% by weight, more preferentially between 50% and 98% by weight, and better still between 70% and 95% by weight, relative to the total weight of the composition (B).

According to a preferred embodiment of the invention, the preparation process according to the invention further comprises:
a treatment step (d₁) comprising the introduction of the particles of element M oxide obtained at the end of step (c) into an alkaline bath of pH 7 to 11, and preferentially of pH 7.5 to 9, and/or
a step (d₂) of calcining the particles of element M oxide obtained at the end of step (c) or at the end of treatment step (d₁).

When the treatment step (d₁) is present:
(i) the treatment lasts preferably between 10 and 600 minutes, more preferentially between 40 and 300 minutes; and/or
(ii) the pH of the alkaline bath varies preferably between 7 and 11, more preferentially between 7.5 and 9; and/or
(iii) the temperature is preferably room temperature, i.e. 25° C.; and/or
(iii) the content of particles of element M oxide obtained at the end of step (c) in the alkaline bath is preferably between from 0.5 to 100 g of particles per litre of alkaline bath, more preferentially between 1 and 10 g of particles per litre of alkaline bath.

When the calcining step (d₂) is present:
(i) the calcining lasts preferably between 60 and 400 minutes, more preferentially between 60 and 180 minutes; and/or
(ii) the temperature ranges preferably from 100° C. to 600° C., more preferentially from 100° C. to 300° C., and more preferentially still from 130° C. to 250° C.

According to one specific embodiment of the invention, the production process further comprises, at the end of step (c), a treatment step (d₁), followed by a calcining step (d₂).

According to one specific embodiment of the invention, the particles of element M oxide obtained by the preparation process according to the invention are doped. According to this embodiment of the invention, composition (A) further comprises one or more precursors of element D, different from element M, with D chosen from fluorine, yttrium, vanadium, scandium, zirconium, hafnium, iron, copper and tungsten.

### The Particles of Element M Oxide

Another subject of the invention is a particle of element M oxide comprising a core (1) and one or more upper coating layers (2) covering said core (1), characterized in that:
(i) the core (1) consists of one or more element M oxides, preferably in the crystalline state;
(ii) said upper coating layer(s) (2) comprise one or more silicon oxides SiO₂ and cover at least 90% of the surface of the core (1), preferably cover the whole of the surface of the core (1);
(iii) said element M is chosen from magnesium, calcium, zinc, copper, iron, zirconium, aluminium, gallium, indium, tin, scandium, yttrium, lanthanum, cerium, praseodymium, promethium, samarium, europium, gadolinium, terbium, dysprosium, holmium, erbium, thulium, ytterbium and lutecium, and
(iv) the (M/Silicon)_{particle} molar atomic ratio is within the range of from 0.1 to 10, preferably from 0.2 to 2, and more preferentially from 0.5 to 1.5.

Preferably, the particle according to the invention comprises a core 1 consisting of element M oxide in the crystalline state. The crystalline state of the core 1 and also its composition may be, for example, determined by a conventional X-ray diffraction method.

Advantageously, the core 1 of the particle according to the invention consists of one or more aggregates of crystalline primary particles of element M oxide. In other words, the core 1 consists of several microcrystals of element M oxide.

Preferably, the particle of element M oxide is obtained by the preparation process of the invention as defined above.

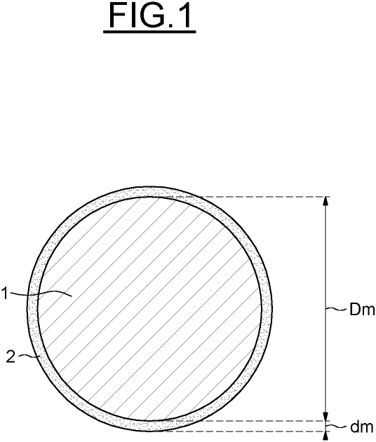

The particle of element M oxide according to FIG. 1 comprises a core 1 of diameter Dₘ, consisting of element M oxide in the crystalline state and comprising one or more aggregates of primary particles of element M oxide.

The particle of element M oxide according to FIG. 1 also comprises an upper coating layer 2 completely covering the surface of the core 1 and having a thickness dm.

The number-average diameter Dₘ of the core 1 may, for example, be determined by transmission electron microscopy (abbreviated to TEM). Preferably, the number-average diameter Dₘ of the core 1 of the particle according to the invention is within the range of from 3 to 1000 nm; more preferentially from 6 to 50 nm, and more preferentially still between 10 and 30 nm.

The particle of element M oxide according to the invention comprises one or more upper coating layers covering at least 90% of the surface of the core 1, and preferably covering the whole of the surface of the core.

The degree of coverage of the core by the upper coating layer(s) may for example be determined by means of a visual analysis of TEM-BF or STEM-HAADF type, coupled to a STEM-EDX analysis.

Each of the analyses is carried out on a statistical number of particles, in particular on at least 20 particles. The particles are deposited on a metal grid made of a metal different from the element M, and from any other metal that forms part of the particles, whether in the core or in the upper coating layer(s). For example, the grid is made of copper (except in the case where it is desired to use copper in the manufacture of the particles).

Visual analyses of the TEM-BF and STEM-HAADF images make it possible, based on the contrast, to deduce whether or not the coating completely surrounds the core of the particle. It is possible, by analysing each of the 20 (or more) images, to deduce a degree of coverage of the core, then, by taking the average, to determine an average degree of coverage.

The STEM-EDX analysis makes it possible to verify that the coating does indeed contain predominantly or exclusively silicon. For this, it is necessary to make measurements (on at least 20 particles), on the edges of the particles. These measurements then reveal the silicon.

The STEM-EDX analysis also makes it possible to verify that the core does indeed contain the element M. For this, it is necessary to make measurements (on at least 20 particles), at the centres of the particles. These measurements then reveal the element M and the silicon.

Preferably, the upper coating layer(s) completely cover the surface of the core.

The number-average thickness dₘ of the upper coating layer(s) may also be determined by transmission electron microscopy. Preferably, the number-average thickness dₘ is within the range of from 1 to 30 nm; more preferentially from 1 to 15 nm and more preferentially still from 1 to 6 nm.

Advantageously, the upper coating layer(s) are amorphous.

The core consists of one or more element M oxides.

Advantageously, the element M is chosen from magnesium, calcium, zinc, copper, iron, zirconium, aluminium, gallium, indium, tin, scandium, yttrium, lanthanum, cerium, praseodymium, promethium, samarium, europium, gadolinium, terbium, dysprosium, holmium, erbium, thulium, ytterbium and lutecium; preferably from magnesium, calcium, zinc, copper, iron, aluminium, tin, lanthanum, cerium and yttrium.

Preferably, the element M oxide thus formed is stable and advantageously has the formula MₓO_{y}, with x and y such that 1 ≤ y/x ≤ 2.

According to one specific embodiment of the invention, the element M is chosen from magnesium and calcium, and the element M oxide thus formed has the formula MO. In other words, according to this embodiment, x=y=1.

According to another specific embodiment of the invention, the element M is chosen from zinc, copper, iron, zirconium, aluminium, gallium, indium, tin, scandium, yttrium, lanthanum, cerium, praseodymium, promethium, samarium, europium, gadolinium, terbium, dysprosium, holmium, erbium, thulium, ytterbium and lutecium; and more particularly from zinc, copper, iron, aluminium, tin, lanthanum, cerium and yttrium.

According to these embodiments, the oxide(s) of element M thus formed are preferably chosen from zinc oxide ZnO, magnesium oxide MgO, calcium oxide CaO, copper oxide CuO, iron oxide Fe₂O₃, aluminium oxide Al₂O₃, cerium oxide CeO₂, lanthanum oxide La₂O₃ and yttrium oxide Y₂O₃; and more preferentially from zinc oxide ZnO, magnesium oxide MgO, calcium oxide CaO, copper oxide CuO and iron oxide Fe₂O₃.

The particle of element M oxide according to the invention comprises the element M and silicon in an (M/Silicon)_{particle} molar atomic ratio for the particle according to the invention.

This ratio corresponds to the amount in moles of element M atoms present in the particle according to the invention on the one hand, to the the amount in moles of silicon atoms present in the particle according to the invention on the other hand.

This ratio can be determined by spectrometry according to one of the following two methods. According to a first method, powder is spread out and an X-ray fluorimetry study is carried out with an X-ray spectrometer to deduce therefrom the metal ratio. According to another method, the particles of the invention are dissolved beforehand in an acid. Then an elemental analysis is carried out on the material obtained by ICP-MS (inductively coupled plasma mass spectrometry) to deduce therefrom the metal ratio.

Preferably, this (M/Silicon)_{particle} molar atomic ratio is within the range of from 0.1 to 10, preferably from 0.2 to 2, and more preferentially from 0.5 to 1.5.

The number-average diameter of the particle according to the invention may also be determined by transmission electron microscopy. Preferably, the number-average diameter of the particle according to the invention is within the range of from 3 to 1000 nm; more preferentially from 10 to 100 nm, and better still from 15 to 70 nm.

Preferably, the BET specific surface area of the particle according to the invention is between 1 m²/g and 350 m²/g; more preferentially between 1 m²/g and 200 m²/g; and even more preferentially between 30 and 100 m²/g.

Preferably, the sum of the content of element M oxide(s) and the content of silicon oxide(s) is at least equal to 99% by weight, relative to the total weight of the core 1 and of the upper coating layer(s) 2.

The particle of element M oxide may optionally further comprise an additional coating layer covering the upper coating layer(s) and comprising at least one hydrophobic organic compound.

The hydrophobic organic compound(s) included in the additional coating layer are more preferentially chosen from silicones, in particular silicones comprising at least one fatty chain; carbon-based derivatives comprising at least 6 carbon atoms, in particular fatty acid esters; and mixtures thereof.

The additional coating layer may be produced via a liquid method or via a solid method. Via a liquid method, the hydroxyl functions of the surface of the particles are reacted with reactive functions of the compound which will form the coating (typically silanol functions of a silicone or the acid functions of carbon-based fatty substance). Via a solid method, the particles are brought into contact with a liquid or pasty compound comprising the hydrophobic substance Then, after contact, the mixture is dried and the mixture is crushed, for example by milling.

Another subject of the invention relates to a composition, preferably a cosmetic composition, comprising one or more particles of element M oxide as described above, and preferably obtained by the preparation process according to the invention.

The composition according to the invention is advantageously an aqueous composition.

The coated particle(s) of element M oxide of the invention may also be in dry form (powder, flakes, plates), as a dispersion or as a liquid suspension or as an aerosol. The coated particle(s) of element M oxide of the invention may be used as is or mixed with other ingredients.

The composition of the invention may be in various galenical forms. Thus, the composition of the invention may be in the form of a powder (pulverulent) composition or of a liquid composition, in the form of a milk, a cream, a paste or an aerosol composition.

The composition according to the invention is in particular a cosmetic composition, i.e. the multilayer material(s) of the invention are in a cosmetic medium. The term "cosmetic medium" means a medium that is suitable for application to keratin materials, notably human keratin materials such as the skin, said cosmetic medium generally consisting of water or of a mixture of water and of one or more organic solvents or of a mixture of organic solvents. Preferably, the composition comprises water, in a content notably of between 5% and 95% by weight relative to the total weight of the composition.

The term "organic solvent" means an organic substance that is capable of dissolving another substance without chemically modifying it. As examples of organic solvents that can be used in the composition of the invention, mention may for example be made of lower C₂—C₆ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

When they are present, the organic solvent(s) are present in proportions preferably between 0.1% and 40% by weight, more preferentially between 1% and 30% by weight and even more particularly between 5% and 25% by weight relative to the total weight of the composition.

The compositions of the invention may contain a fatty phase and may be in the form of direct or inverse emulsions.

The content of the particle(s) of element M oxide, present in the composition of the invention, ranges preferably from 0.1% to 40% by weight, more preferentially from 0.5% to 20% by weight, better still from 1% to 10% by weight and more preferentially still from 1.5% to 5% by weight, with respect to the total weight of the composition.

According to one specific embodiment of the invention, the composition according to the invention may also be in the form of an anhydrous composition, for instance in the form of an oil. The term "anhydrous composition" is intended to mean a composition containing less than 2% by weight of water, preferably less than 1% by weight of water, and even more preferentially less than 0.5% by weight of water relative to the total weight of the composition, or even a composition that is free of water. In this type of composition, the water possibly present is not added during the preparation of the composition, but corresponds to the residual water provided by the mixed ingredients.

The composition according to the invention may be prepared according to the techniques that are well known to those skilled in the art. It may in particular be in the form of a simple or complex emulsion (oil-in-water, or abbreviated to O/W, water-in-oil or W/O, oil-in-water-in-oil or O/W/O, or water-in-oil-in-water or W/O/W), such as a cream, a milk or a cream gel, or else in powder form or in the form of an aerosol composition.

Another subject of the invention is the composition according to the invention, preferably a cosmetic composition, for use for protecting the skin, in particular human skin, against visible radiation (i.e. wavelengths between 400 nm and 800 nm) and/or ultraviolet radiation (i.e. wavelengths between 100 nm and 400 nm), UV-A radiation (i.e. wavelengths between 320 nm and 400 nm) and/or UV-B radiation (i.e. wavelengths between 280 nm and 320 nm). The compositions according to the invention make it possible to screen out solar radiation efficiently, they are broad-spectrum, in particular for UV-A radiation (including long-wave UV-A radiation), while being particularly stable over time under UV exposure.

The composition according to the present invention may optionally comprise one or more additional UV-screening agents, other than the particle of element oxide M according to the invention, chosen from hydrophilic, lipophilic or insoluble organic UV-screening agents and/or one or more mineral pigments. It will preferentially be constituted of at least one hydrophilic, lipophilic or insoluble organic UV-screening agent.

Another subject of the invention is the use of the particles of element M oxide as described above, and preferably obtained by the process according to the invention:
for formulating cosmetic or pharmaceutical compositions, in particular intended to protect the skin, in particular human skin, against visible and/or ultraviolet radiation or to modify the appearance of the skin, in particular human skin,
for formulating paints, varnishes and/or stains, or
for manufacturing a coating for electronic devices or products, notably for obtaining moisture-resistant electronic components.

### Application Process

Another subject of the invention is a process for treating keratin materials, notably human keratin materials such as the skin, by application to said materials of a composition as defined previously, preferably by 1 to 5 successive applications, leaving to dry between the layers, the application(s) being sprayed or otherwise.

The compositions of the invention may be used in single application or in multiple application. When the compositions of the invention are intended for multiple application, the content of particles of element M oxide of the invention is generally lower than in compositions intended for single application.

For the purposes of the present invention, the term "single application" means a single application of the composition, this application possibly being repeated several times per day, each application being separated from the next by one or more hours, or an application once a day, depending on the need.

For the purposes of the present invention, the term "multiple application" means application of the composition repeated several times, in general from 2 to 5 times, each application being separated from the next by a few seconds to a few minutes. Each multiple application may be repeated several times per day, separated from the next by one or more hours, or each day, depending on the need.

They may also be connected application methods, such as a saturated single application, i.e. the single application of a cosmetic composition with a high concentration of particles of element M oxide coated with silicon oxide according to the invention, or else with multiple applications of cosmetic composition (less concentrated) comprising one or more particles of element M oxide coated with silicon oxide according to the invention. In the case of multiple applications, several successive applications of cosmetic compositions comprising one or more particles of element M oxide coated with silicon oxide of the invention may be repeated with or without a delay between the applications.

According to one embodiment of the invention, the multiple application is performed on the keratin materials with a drying step between the successive applications of the cosmetic compositions comprising particle(s) of element M oxide coated with silicon oxide according to the invention. The drying step between the successive applications of the cosmetic compositions comprising one or more particles of element M oxide coated with silicon oxide according to the invention may be performed in the open air or artificially, for example with a hot air drying system such as a hairdryer.

Another subject of the invention is the use of one or more particles of element M oxide coated with silicon oxide according to the invention as defined above as UVA and UVB screening agent to protect keratin materials, notably human keratin materials, such as the skin.

The examples that follow serve to illustrate the invention without, however, being limiting in nature.

### Examples

### Example 1

1.1. Firstly, a composition (A) of zinc naphthenate (500 mM) in xylene was prepared. Next, zinc oxide particles coated with silicon dioxide P1 were then prepared using an FSP process according to the invention comprising the injections of composition (A) and of a composition (B) comprising hexadimethyldisiloxane and ethanol in a proportion of 3: 1.

The parameters of the preparation process are the following:

- ratio (composition (A) / O₂) = 5/7, i.e. 5 mL/min of liquid and 7 L/min of gas (O₂). To adjust the oxygen flow rate, φ = 0.48 is used.

In this process, a 40 cm high quartz tube is used. Furthermore, nitrogen first is bubbled through the composition (B). When the composition (B) is injected, the stream of nitrogen heated to 25° C. is adjusted in order to enable the evaporation of the hexadimethyldisiloxane (HMDSO) and so that the ratio (Zn/Si)_{injected}ratio = 1.

1.2. The Raman spectrum of these particles showed that the coating has a specific structure, the silicon atoms being mostly in "4-membered ring" form. The peak of the Si—O "3-membered ring" also being present, but with a lower intensity and smaller peak area.

### 1.3 Evaluation of the Water Resistance

An aqueous suspension S1 was prepared from particles P1 and water in a content of 100 mg of P1/L of water. The suspension S1 thus obtained was then placed in an ultrasound bath for 10 min at a power of 20 W.

Then, a fraction of the suspension S1 was brought to pH = 5 by means of a nitric acid solution. The content of Zn²⁺ ions present in the suspension as a function of time, and relative to the amount of zinc introduced, was then measured by means of a conventional anodic stripping voltammetry method.

The results have been collated in the table below:

| Suspensions | Content of Zn²⁺ (% ions released in a litre of water) | | | | |
|---|---|---|---|---|---|
| | at t₀ | at t₀+1 h | at t₀+2 h | at t₀+3 h | at t₀+4 h |
| S1 at pH 5 | 0 | 1.2 | 2.2 | 4.6 | 6.2 |

t₀ corresponds to the first measurement carried out less than 10 min after the end of the ultrasound bath.

It should be noted that the coated zinc oxide particles P1 obtained according to the preparation process of the invention have a good water resistance, even at acid pH.

### Example 2

2.1. Zinc oxide particles coated with silicon dioxide P2 were prepared according to the process described in example 1. The particles P2 thus obtained were then separated into two groups P2a and P2b. The particles P2a underwent a post-treatment according to the protocol below, whilst the particles P2b did not undergo post-treatment.

Protocol of the post-treatment for the particles P2a:

After collecting the particles obtained by the process, two treatments are carried out:
a treatment step (d₁), for 120 minutes, comprising introducing the particles of element M oxide obtained at the end of step (c) into an alkaline bath of pH 7.5 and at a temperature of 25° C. and with a ratio of 5 g of particles per 1 litre of alkaline bath (mixture of sodium hydroxide and water); and
a step of calcining (d₂), for 120 minutes at 200° C., the particles of element M oxide obtained at the end of step (d₁).

2.2. It was observed that the zinc oxide particles P2a and P2b thus obtained were crystalline and coated with silicon dioxide. The (Zn/Si)_{particle} atomic ratio is 1 and the BET specific surface area of the particles is 116 m²/g for the particles P2a and and 74 m²/g for the particles P2b. The particles moreover have a number-average diameter equal to 40 nm.

A water resistance test equivalent to example 1 was carried out and shows that the particles that underwent the FSP treatment and the post-treatment (P2a), have an even higher water resistance.

An aqueous suspension S1' was prepared from particles P2a and water in a content of 100 mg of P2a/L of water. The suspension S1' thus obtained was then placed in an ultrasound bath for 10 min at a power of 20 W.

Then, a fraction of the suspension S1' was brought to pH = 5 by means of a nitric acid solution. The content of Zn²⁺ ions present in the suspension as a function of time, and relative to the amount of zinc introduced, was then measured by means of a conventional anodic stripping voltammetry method.

The results have been collated in the table below:

| Suspensions | Content of Zn²⁺ (% ions released in a litre of water) | | | | |
|---|---|---|---|---|---|
| | at t₀ | at t₀+1 h | at t₀+2 h | at t₀+3 h | at t₀+4 h |
| S1 at pH 5 (example 2) | 0 | 0.4 | 0.8 | 1.2 | 1.4 |
| S1 at pH 5 (example 1) | 0 | 1.2 | 2.2 | 4.6 | 6.2 |

### 2.3 Evaluation of the Water Resistance

Two aqueous suspensions S2a and S2b were prepared from particles P2a and P2b and water in a content of 100 mg of particles per litre of water at pH 8.2.

An aqueous suspension S3 was moreover prepared from zinc oxide particles sold under the reference Z-COTE HP1 (Oxide and Triethoxycaprylylsilane) by the company BASF and water in a content of 100 mg of commercial particles per litre of water. In other words, the particles Z-COTE HP1 are coated with a layer of triethoxycaprylylsilane, thus giving them a hydrophobic property and protection from water. The BET specific surface area of these particles is 15.8 m²/g.

The suspensions S2a, S2b and S3 thus obtained were then placed in a ultrasound bath for 10 min at a power of 20 W.

The content of Zn²⁺ ions present in each of the suspensions S2a, S2b and S3 as a function of time was then measured at various moments by means of a conventional anodic stripping voltammetry method for each suspension.

The results have been collated in the table below:

| Suspensions | Content of Zn²⁺ (concentration of ions released in a litre of water (ppb)) | | | |
|---|---|---|---|---|
| | at t₀ | at t₀+2 h | at t₀+24 h | at t₀+48 h |
| S2a at pH 8.2 (invention) | 0 | 14 | 19 | 25 |
| S2b at pH 8.2 (invention) | 0 | 90 | 105 | 135 |
| S3 at pH 8.2 (comparative) | 0 | 790 | 1550 | 2450 |

t₀ corresponds to the first measurement carried out less than 10 min after the end of the ultrasound bath.

he Raman spectrum of S2a is similar to that of S1, i.e. with a peak corresponding to the silicon atoms in "4-membered ring" form. The peak of the Si—O "3-membered ring" also being present, but with a lower intensity and smaller peak area.

It should be noted that the coated zinc oxide particles P2a and P2b obtained according to the process of the invention have a better water resistance than the commercial zinc oxide particles, despite an especially high BET specific surface area (116 m²/g and 74 m²/g for the particles P2a and P2b versus 15.8 m²/g for the commercial compound). This resistance is even more improved since the particles formed at the end of the process underwent an alkaline post-treatment (P2b).

Furthermore, it should be noted that the coated zinc oxide particles P2a and P2b according to the invention have a screening power identical to that of the commercial zinc oxide particles. The zinc oxide particles of the invention make it possible to obtain a better water resistance, a better transparency in the visible spectrum, while retaining good UVA-screening properties.

### Example 3

A composition (C) of magnesium naphthanate (C₂₂H₁₄O₄Mg) (500 mM) in xylene was prepared.

Uncoated magnesium oxide particles P4 were then prepared using a conventional FSP preparation process Prep 1 with the pre-prepared composition (C) (outside the invention).

Next, magnesium oxide particles coated with silicon dioxide P5 were then prepared using a preparation process Prep 2 according to the invention with the same composition (C) and a composition (B) comprising hexadimethyldisiloxane and ethanol in a proportion of 3: 1 (invention).

The parameters of the Prep 1 process are the following:

- ratio (composition (C) / O₂) = 5 mL/min of liquid and 7 L/min of gas (O₂). To adjust the oxygen flow rate, φ = 0.48 is used.

The parameters of the Prep 2 process are the following:

- ratio (composition (C) / O₂) = 5/7, i.e. 5 mL/min of liquid and 7 L/min of gas (O₂). To adjust the oxygen flow rate, φ = 0.48 is used.

In this Prep 2 process, a 40 cm high quartz tube is used. Furthermore, nitrogen is first bubbled through the composition (B). When the composition (B) is injected, the stream of nitrogen heated to 25° C. is adjusted in order to enable the evaporation of the hexadimethyldisiloxane (HMDSO) and so that the (Zn/Si)_{injected}ratio = 1.

The particles P5 thus obtained were then separated into two groups P5a and P5b. The particles P5a underwent a post-treatment according to the protocol below, whilst the particles P5b did not undergo post-treatment.

Protocol of the post-treatment for the particles P5a:

After collecting the particles obtained by the process, two treatments are carried out:
a treatment step (d₃) for 120 minutes, comprising introducing the particles of element M oxide obtained at the end of step (c) into an alkaline bath of pH 8.5 and at a temperature of 25° C. and with a ratio of 5 g of particles per 1 litre of alkaline bath (mixture of sodium hydroxide and water); and
a step of calcining (d₄), for 120 minutes at 200° C., the particles of element M oxide obtained at the end of step (d₃).

Once the particles had been prepared, it was observed that the magnesium oxide particles obtained were crystalline.

Furthermore, the particles obtained according to process Prep 2 according to the invention are coated with silicon dioxide and have an (Mg/Si)_{particle} atomic ratio of 1.

The BET specific surface area of the particles according to process Prep 2 is 24 m²/g.

The particles have a size: 40-90 nm

Next, the resistance of the three P4, P5a and P5b particle samples are evaluated.

For this:
0.1 g of the P4 sample is placed in a 2.5 L bath of water acidified by hydrochloric acid (pH = 5), i.e. 1 mM as MgO equivalent
0.2 g of each of the P5a and P5b samples is placed in a 2.5 L bath of water acidified by hydrochloric acid (pH = 5), i.e. 1 mM as MgO equivalent.

After stirring for 30 min with a magnetic stirrer, the amount of solid matter collected is filtered and weighed, then a content of "collected solid matter" (filtered mass/initial mass) is deduced therefrom.

In the case of P4 (uncoated MgO particles), no collected solid matter is found.

In the case of P5a, the solid matter collected is 93%.

In the case of P5b, the solid matter collected is 78%.

### Example 4

A composition (D) of titanium naphthenate (500mM) in xylene was prepared.

Next, titanium oxide particles coated with silicon dioxide P6 were then prepared using an FSP process according to the invention comprising the injections of said composition (D) and of a composition (B) comprising hexadimethyldisiloxane and ethanol in a proportion of 3: 1 (particles P6).

The parameters of the preparation processes are the following:

ratio (composition (D) / O₂) = 5/7, i.e. 5 mL/min of liquid and 7 L/min of gas (O₂). To adjust the oxygen flow rate, φ = 0.48 is used.

In these processes, a 40 cm high quartz tube is used. Furthermore, nitrogen is first bubbled through the composition (B). When the composition (B) is injected, the stream of nitrogen heated to 25° C. is adjusted in order to enable the evaporation of the hexadimethyldisiloxane (HMDSO) and so that the (Ti/Si)_{injected} ratios are equal to 1.

### Example 5

5.1. Zinc oxide particles coated with silicon dioxide P1 (invention) were prepared according to the process described in example 1.

Zinc oxide particles coated with silicon dioxide P7 (comparative) were prepared at the same time using the same process comprising the injections of composition (A) described in example 1 and of a composition (B1) comprising tetraethoxysilane and ethanol in a proportion of 3: 1.

Zinc oxide particles coated with silicon dioxide P8 (comparative) were also prepared using the same process comprising the injections of composition (A) described in example 1 and of a composition (B2) comprising only hexadimethyldisiloxane.

The parameters of the preparation processes are the following:

ratio (composition (A) / O₂) = 5/7, i.e. 5 mL/min of liquid and 7 L/min of gas (O₂). To adjust the oxygen flow rate, φ = 0.48 is used.

In these processes, a 40 cm high quartz tube is used. Furthermore, nitrogen is first bubbled through the compositions (B), (B 1) and (B2). When these compositions (B), (B1) and (B2) are injected, the stream of nitrogen heated to 25° C. is adjusted in order to enable the evaporation of the hexadimethyldisiloxane (HMDSO) or of the tetraethoxyslane and so that the (Zn/Si)_{injected} ratio = 1.

5.2. The Raman spectra of the particles P7 and P8 show that, unlike the Raman spectrum of the particles P1, the peak corresponding to the particular structure according to which the silicon atoms are in the "4-membered ring" form, is of low intensity (P7) or too broad (P8). These results show that the "4-membered ring" form is formed only moderately in the case of P7, and is not formed in the comparative particles P8.

### 5.3 Evaluation of the Water Resistance

Aqueous suspensions S1, S7 and S8 were prepared from particles P1, P7 and P8 and water in a content of 100 mg of particles per litre of water. The suspensions S1, S7 and S8 thus obtained were then placed in a ultrasound bath for 10 min at a power of 20 W.

Then, a fraction of the suspensions S1, S7 and S8 was brought to pH = 5 by means of nitric acid.

The content of Zn²⁺ ions present in each of the suspensions S1, S7 and S8 as a function of time, and relative to the amount of zinc introduced, was then measured by means of a conventional anodic stripping voltammetry method for each suspension.

The results have been collated in the table below:

| Suspensions | Content of Zn²⁺ (% ions released in a litre of water) | | | | |
|---|---|---|---|---|---|
| | at t₀ | at t₀+1 h | at t₀+2 h | at t₀+3 h | at t₀+4 h |
| S1 (invention) at pH 5 | 0 | 1.2 | 2.2 | 4.6 | 6.2 |
| S7 (comparative) at pH 5 | 0 | 16 | 18 | 20 | 23 |
| S8 (comparative) at pH 5 | 0 | 55 | 62 | 66 | 68 |

t₀ corresponds to the first measurement carried out less than 10 min after the end of the ultrasound bath.

It should be noted that the coated zinc oxide particles P1 obtained according to the preparation process of the invention, using a silicon precursor comprising at least two silicon atoms and Si-carbon covalent bonds in a specific solvent, have excellent water resistance.

The coated particles P7 obtained with a specific silicon precursor that does not comprise two silicon atoms, in a specific solvent, give good water resistance.

Conversely, the particles P8 obtained with a specific silicon precursor that does comprise two silicon atoms, but with no specific solvent, does not give good water resistance.

Thus, it is concluded that it is not sufficient to produce a silica-based coating in order to obtain a water protection effect.

1. Process for preparing coated particles of element M oxide, characterized in that it comprises at least the following steps:
a) preparing a composition (A) by adding one or more element M precursors to one or more combustible solvents; then
b) in a flame spray pyrolysis device, forming a flame by injecting the composition (A) and an oxygen-containing gas until aggregates of element M oxide are obtained; and
c) injecting into the flame a composition (B) comprising one or more silicon precursors and one or more polar protic solvents other than water until an inorganic coating layer containing a silicon oxide is obtained on the surface of said aggregates of element M;
it being understood that:
said element M is chosen from alkali metals from column 1, alkaline-earth metals from column 2 and the elements from columns 3 to 16 of the Periodic Table of the Elements, and elements from the family of lanthanides, and
the silicon precursor(s) comprise at least two silicon atoms and several Si-carbon covalent bonds.

2. Process according to claim 1, characterized in that the element M is chosen from magnesium, calcium, zinc, copper, iron, titanium, zirconium, aluminium, gallium, indium, tin, scandium, yttrium, lanthanum, cerium, praseodymium, promethium, samarium, europium, gadolinium, terbium, dysprosium, holmium, erbium, thulium, ytterbium and lutecium; preferably from magnesium, calcium, zinc, copper, iron, titanium, aluminium, tin, lanthanum, cerium and yttrium.

3. Process according to claim 1,, characterized in that the element M precursor(s) comprise one or more atoms of element M optionally complexed with one or more ligands containing at least one carbon atom; preferably said ligand(s) are chosen from the following groups: acetate, (C₁-C₆)alkoxylate, (di)(C₁C₆)alkylamino, and arylate such as naphthalate or naphthenate.

4. Process according to claim 1, characterized in that the element M oxide has the formula MₓO_{y}, with x and y such that 1 ≤ y/x ≤ 2.

5. Process according to claim 1, characterized in that the content of element M precursor in the composition (A) is between 1% and 60% by weight, preferably between 15% and 30% by weight, relative to the total weight of the composition (A).

6. Process according to claim 1, characterized in that the flame formed in step (b) and maintained in step (c) is, at the outlet of the tube transporting the composition (B), at a temperature between 200° C. and 600° C.; preferably between 300° C. and 400° C.

7. Process according to claim 1., characterized in that the silicon precursor(s) in the composition (B) comprise at least three silicon atoms and several Si-carbon covalent bonds; preferably the silicon precursor(s) are chosen from hexadimethyldisiloxane, 1,2-bis(triethoxysilyl)ethane, 1,2-bis(trimethoxysilyl)ethane, and mixtures thereof.

8. Process according to claim 1, characterized in that the (MISilicon)injected molar atomic ratio is within the range of from 0.1 to 10, preferably from 0.2 to 2, and more preferentially from 0.5 to 1.5.

9. Process according to claim 1, characterized in that the polar protic solvent(s) other than water in the composition (B) are chosen from (C1-C8)alkanols, preferably the solvent is ethanol.

10. Process according toclaim 1,, characterized in that the content of silicon precursor in the composition (B) is between 1% and 60% by weight, preferably between 5% and 30% by weight, relative to the total weight of the composition (B).

11. Process according toclaim 1., characterized in that it further comprises a treatment step (d1) comprising the introduction of the particles of element M oxide obtained at the end of step (c) into an alkaline bath of pH 7 to 11, and/or a step of calcining (d2) the particles of element M oxide obtained at the end of step (c) or at the end of the treatment step (d1).

12. Particle of element M oxide comprising a core (1) and one or more upper coating layers (2) covering said core (1), characterized in that:
(i) the core (1) consists of one or more element M oxides, preferably in the crystalline state;
(ii) said upper coating layer(s) (2) comprise one or more silicon oxides and cover at least 90% of the surface of the core (1), preferably cover the whole of the surface of the core (1);
(iii) said element M is chosen from magnesium, calcium, zinc, copper, iron, zirconium, aluminium, gallium, indium, tin, scandium, yttrium, lanthanum, cerium, praseodymium, promethium, samarium, europium, gadolinium, terbium, dysprosium, holmium, erbium, thulium, ytterbium and lutecium, and
(iv) the (M/Silicon)particle molar atomic ratio is within the range of from 0.1 to 10, preferably from 0.2 to 2, and more preferentially from 0.5 to 1.5.

13.Particle according to characterized in that it is obtained by the process as defined in any one of claims 1 to 11.

14. Particle according to claim 12 , characterized in that the element M oxide(s) constituting the core (1) are stable; preferably the element M oxide(s) are chosen from zinc oxide ZnO, magnesium oxide MgO, calcium oxide CaO, copper oxide CuO, iron oxide Fe₂O₃, aluminium oxide Al₂O₃, cerium oxide CeO₂, lanthanum oxide La₂O₃ and yttrium oxide Y₂O₃; and more preferentially from zinc oxide ZnO, magnesium oxide MgO, calcium oxide CaO, copper oxide CuO, and iron oxide Fe₂O₃.

15. Particle according to claim 12characterized in that the sum of the content of element M oxide(s) and the content of silicon oxide(s) is at least equal to 99% by weight, relative to the total weight of the core (1) and of the upper coating layer(s) (2).

16. Particle according to claim 12 characterized in that the number-average diameter Dₘ of the core (1), determined by transmission electron microscopy (TEM), is within the range of from 3 to 1 000 nm, preferably from 6 to 50 nm, and more preferentially from 10 to 30 nm.

17. Particle according to claim 12, characterized in that the number-average thickness dₘ of the upper coating layers (2), measured by transmission electron microscopy (TEM), is within the range of from 1 to 30 nm, preferably from 1 to 15 nm, and more preferentially from 1 to 6 nm.

18. Particle according to claim 12, characterized in that the number-average diameter of the particle, determined by transmission electron microscopy (TEM), is within the range of from 3 to 1 000 nm, preferably from 10 to 100 nm, and more preferentially from 15 to 70 nm.

19. Composition comprising one or more particles of element M oxide as obtained by the process defined in claim 1, and/or as defined in any one of claims 12 to 18.

20. Composition as defined in claim 19 for use for protecting the skin, preferably human skin, against visible and/or UV-A and/or UV-B ultraviolet radiation.

21. Use of the particles of element M oxide claim 1:
for formulating cosmetic or pharmaceutical compositions, in particular intended to protect the skin, in particular human skin, against visible and/or ultraviolet radiation or to modify the appearance of the skin, in particular human skin,
for formulating paints, varnishes and/or stains, or
for manufacturing a coating for electronic devices or products, notably for obtaining moisture-resistant electronic components.
